(19) United States
(12) Patent Application Publication
(10) Pub. No.: US 2015/0031582 A1
(43) Pub. Date: Jan. 29, 2015

(54) Volatile Organic Gases as Bioindicators for Transplant Rejection

(71) Applicant: University of Houston System, Houston, TX (US)

(72) Inventors: Dong Cai, Houston, TX (US); Zhifeng Ren, Houston, TX (US); Xian C. Li, Houston, TX (US); Renard L. Thomas, Houston, TX (US)

(73) Assignee: UNIVERSITY OF HOUSTON SYSTEM, Houston, TX (US)

(21) Appl. No.: 14/444,863

(22) Filed: Jul. 28, 2014

Related U.S. Application Data
(60) Provisional application No. 61/859,606, filed on Jul. 29, 2013.

(51) Int. Cl. G01N 33/497; G01N 33/493

(52) U.S. Cl. CPC G01N 33/497; G01N 33/493; G01N 2570/00; G01N 2800/245
USPC 506/12; 436/141; 436/129; 436/120; 436/130; 436/128; 422/82.01; 506/39

(57) ABSTRACT

A method comprising (a) obtaining one or more biological samples from a subject wherein the subject has undergone an organ transplant; (b) determining the amount and type of one or more volatile organic compounds in the biological sample; and (c) correlating the amount of volatile organic compounds to a degree of transplant rejection. A device comprising a plurality of sensors configured to detect a plurality of volatile organic compounds in a biological sample of a subject having undergone an organ transplant.

### CROSS-REFERENCE TO RELATED APPLICATIONS

This application claims priority to U.S. Provisional Patent Application No. 61/859,606 filed on Jul. 29, 2013, and titled “Volatile Organic Gases as Bioindicators for Transplant Rejection” incorporated herein by reference in its entirety.

### STATEMENT REGARDING FEDERALLY SPONSORED RESEARCH OR DEVELOPMENT

Not applicable.

### BACKGROUND

1. Field of the Disclosure

The present disclosure relates to detecting complications resultant from organ transplantation. More specifically, the present disclosure relates to a method of diagnosing transplant-organ rejection.

2. Background of the Technology

Generally, organ transplantation is the final treatment for end-state organ failures in humans. All recipients must receive immunosuppressant therapy to prevent rejection, at the expense of increased risk of other complications, including disease. However, between about 20 and about 40 percent of organ recipients experience at least one episode of transplant rejection during the first year, particularly after heart transplantation. A recipient's prognosis declines significantly due to the rejection of the transplanted organ. Most deaths in this period are due to acute rejection or immunosuppression-related infections. Thus, the success of transplantation and the recipient's prognosis are dependent on intensive testing procedures for rejection management and prevention.

In the specific example of heart transplantation, the primary method of rejection analysis and management currently is endo-myocardial biopsy. However, biopsy is an invasive procedure involving the extraction of heart tissue. The tissue then requires subjective inspection and judgment to predict rejection. Therefore, biopsy increases the risk of complications, morbidity, and mortality, and may not be accurate. Still further, rejection of the transplanted organ occurs without acute or obvious symptoms in a many cases.

The search for rejection biomarkers has investigated the genes representing the molecular pathways with the AlloMap test, the donor DNA molecules released from the allograft as the organ-specific signature, as well as the development of anti-donor human-leukocyte-antigen (HLA) antibodies in the recipient. Several testing procedures also have been proposed, including magnetic resonance imaging; antibody imaging; echocardiography; and use of serum markers such as troponin I, troponin T, creatine kinase-MB fraction and C-reactive protein, yet none have yielded diagnostically-satisfactory results desired for early diagnosis.

In order to improve the prognosis of transplant recipients, a non-invasive, accurate and sensitive detection method, is desired for early diagnosis of rejection.

### BRIEF SUMMARY OF THE DISCLOSURE

Disclosed herein is a method comprising (a) obtaining one or more biological samples from a subject wherein the subject has undergone an organ transplant; (b) determining the amount and type of one or more volatile organic compounds in the biological sample; and (c) correlating the amount of volatile organic compounds to a degree of transplant rejection.

Also disclosed herein is a device comprising a plurality of sensors configured to detect a plurality of volatile organic compounds in a biological sample of a subject having undergone an organ transplant.

### BRIEF DESCRIPTION OF THE DRAWINGS

For a detailed description of the exemplary embodiments disclosed herein, reference will now be made to the accompanying drawings in which:

FIG. 1 schematically illustrates a transplant recipient and associated volatile organic compounds (VOC's).

FIG. 2 graphically illustrates a conventional analysis of VOCs in urine.

### DETAILED DESCRIPTION

Disclosed herein are methods, devices and compositions for the detection of a physiological condition and/or physiological state. In an embodiment, the methods disclosed herein are noninvasive methods for the assessment of the physiological condition of a subject. The subject may be experiencing or predisposed to experiencing a medical condition, dysfunction or disorder which are collectively referred to herein as an “undesirable physiological state.” Herein the term “noninvasive” refers to a procedure not requiring the introduction of instruments into the body of the subject. The term “subject,” as used herein, comprises any and all organisms and includes the term “patient.” In an embodiment, the subject is a human or any other animal. In an embodiment, the method comprises obtaining at least one biological sample from the subject and analyzing the sample for the presence of one or more bioindicators. Herein the term “biological sample” or “sample” refers to a material obtained from the body of the subject and includes, without limitation, biological fluids such as urine, saliva, and blood, including derivatives of blood, e.g. plasma, and serum and biological gases such as breath. Herein the term “bioindicators” refers materials whose level of expression in the biological sample obtained from the subject experiencing or predisposed to experiencing an undesirable physiological state (e.g., transplant rejection event) differs as compared to the biological sample obtained from a subject not experiencing or not predisposed to experiencing an undesirable physiological state (e.g., control or “healthy” subject). Herein “predisposed” refers to the increased likelihood of the subject having the transplantation rejection event when compared to the subject's physiological state post-transplantation in the absence of any indication of transplant rejection.

In an embodiment, bioindicators of an undesirable physiological state (e.g., transplant rejection) are VOC's of the type disclosed herein. Herein “volatile” has its traditional meaning, which is a substance that is easily evaporated at room temperature. As understood by one of ordinary skill in the art, a VOC may have some portion of the material in the gaseous state and some portion in the liquid state simultaneously. It is contemplated that detection of the bioindicators of the undesirable physiological process (e.g., transplant rejection) not only permits the identification of a subject having the undesirable physiological state, but may also provide a means to identify a subject predisposed to developing this condition, ideally before physical symptoms manifest. If possible, such early detection would allow for improved patient care and could possibly prevent or mitigate the adverse events associated with the undesirable physiological state. Hereinafter the disclosure will focus on one or more transplant rejection events as the undesirable physiological state where transplant rejection refers to when transplanted tissue is rejected by the recipient's immune system, which destroys the transplanted tissue.

In an embodiment, a method for the detection of a transplant rejection event comprises obtaining a biological sample from a subject. The subject may be further characterized as having undergone at least one organ transplantation procedure where at least a portion of a diseased or dysfunctional organ has been replaced with at least a portion of a healthy organ from a donor with a compatible tissue type. In an aspect of this disclosure, the transplantation procedure comprises an allotransplantation wherein the donor is genetically non-identical. In yet another aspect, the transplantation procedure is a cardiac allograft. A biological sample may be obtained from the subject having undergone the cardiac allograft. For example, the biological sample may be a urine sample. According to a method of the present disclosure, urine samples provide a concentrated aqueous solution of metabolites filtered from the blood by the kidneys and bioindicators of the type disclosed herein may comprise at least a portion of the metabolites.

The urine sample may be conveniently collected from a human subject using a suitable container with a sufficient volume capacity. In one embodiment, a urine container may contain a cap to prevent spilling and a means to allow the collected urine sample to be transported. The urine sample may be stored under appropriate conditions that are compatible with the methodologies of the present disclosure. In one embodiment, urine is collected as first void urine (i.e., in the morning). In another embodiment, urine may be collected during daytime or before bedtime.

In one embodiment, the urine sample may be processed to facilitate detection of the disclosed bioindicators. In some embodiments, the urine sample may be turbid which may be the result of any number of a physiological processes (e.g., dehydration, bacterial infection, etc. . . . ). Potential crude debris present in a turbid urine sample may be cleared prior to further utilization of the sample. For example, collected urine samples may simply be passed through a cloth, paper, tissue, filter and the like. The collected urine sample, having been processed for detection of the bioindicators disclosed herein is termed the treated urine sample (TUS).

In an embodiment, the biological sample is breath. Breath samples may be obtained utilizing a breath collector. The breath collector may be used to increase sensing sensitivities either by concentrating the breath analytes to be detected or by dehumidifying the patient's breath prior to analyzing. This allows for increased resolution in discriminating between different breath samples. In some embodiments, the breath sample may be concentrated prior to analysis for bioindicators such as VOCs. Any suitable method for concentrating the breath may be utilized, such as solid phase microextraction, sorbent tubes, and cryogenic concentration.

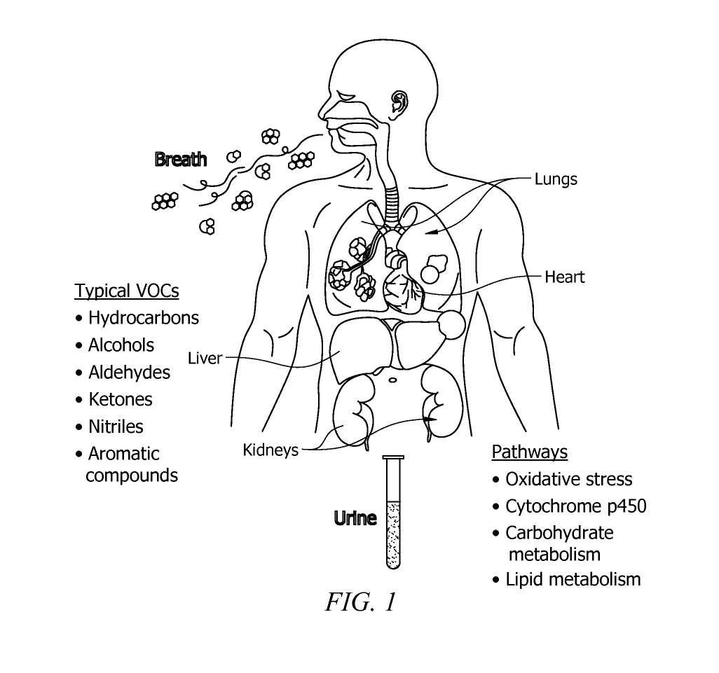

In an embodiment, the biological sample (e.g., TUS or breath) is subjected to one or more methodologies for the detection of one or more bioindicators, for example, one or more VOCs. For example, the biological sample may subjected to one or more devices which function to identify and/or quantitate the amount of VOCs present within the sample. Such devices are disclosed in more detail below. Without being limited by any theory, the VOCs produced during allograft rejection is thought to be the downstream result of oxidative stress associated with the increased generation of reactive oxygen species (ROS) in the myocardium. The ROS are proposed to degrade cellular membranes by lipid peroxidation of polyunsaturated fatty acids which may produce VOCs of the type disclosed herein. Thus VOCs are either end products or by-products of various metabolic pathways, particularly related oxidative stress, cytochrome p450 activity, carbonhydrate metabolism and lipid metabolism as depicted in FIG. 1.

VOCs that may be produced during a transplant rejection comprise hydrocarbons, alcohols, sulfides, sulfoxides, sulfones, aldehydes, ketones, nitriles, aromatics, organic acids, nitrates, thromboxane, neopterin, n-acetylaspartate, myo-inositol, creatine or combinations thereof. The present disclosure contemplates that any VOC that correlates with a transplant rejection event may be monitored and utilized to predict and/or diagnose a transplant rejection event.

In an embodiment, the VOCs comprise hydrocarbons, alternatively alkanes, alternatively methylated alkanes. The term “hydrocarbon” is used herein in accordance with the definition specified by IUPAC and refers to a compound containing only carbon and hydrogen. In an embodiment, the VOCs comprise aliphatic hydrocarbons such as and without limitation C₁ to C₂₀ aliphatic hydrocarbons, alternatively C₂ to C₂₀ hydrocarbons, alternatively C₃ to C₂₀ aliphatic hydrocarbons; alternatively C₄ to C₁₅ aliphatic hydrocarbons; or alternatively, C₅ to C₁₀ aliphatic hydrocarbons. The aliphatic hydrocarbons may be cyclic or acyclic and/or may be linear or branched, unless otherwise specified. Non-limiting examples of aliphatic hydrocarbons that may be utilized as bioindicators for a transplant rejection event singly or in any combination include methane, ethane, propane, iso-butane, n-butane, butane, pentane (n-pentane or a mixture of linear and branched C₅ acyclic aliphatic hydrocarbons), hexane (n-hexane or mixture of linear and branched C₆ acyclic aliphatic hydrocarbons), heptane (n-heptane or mixture of linear and branched C₇ acyclic aliphatic hydrocarbons), octane (n-octane or a mixture of linear and branched C₈ acyclic aliphatic hydrocarbons), or combinations thereof.

Further, in certain instances, the VOCs comprise modified alkanes, such as methylated alkanes. Examples of VOCs that may be analyzed to predict and/or diagnose a transplant rejection event include without limitations 2-methylpropane, 5-methyloctadecane, 6-methyloctadecane, 2-methylheptadecane, 2-methylheptane, 3-methylundecane, 2-methyloctadecane, 2-methylhexadecane, or combinations thereof.

In an aspect, the VOCs comprise organic acids. For example and without limitation, the VOCs may comprise hippuric acid, methyl succinic acid, mandelic acid, muconic acid, fuoric acid, butyric acid, hydroxybutryic acid or derivatives or combinations thereof. In an embodiment, a VOC utilized for the diagnosis and/or prediction of a transplant rejection event is hippuric acid or methylhippuric acid. Hippuric acid also known as benzoyl amidoacetic acid is depicted as Formula I.

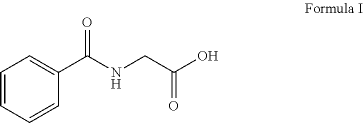 [SMILES: O=C(O)CNC(=O)c1ccccc1]

In an embodiment, the VOC comprises sulfides. For example and without limitation the VOCs may comprise hydrogen sulfide, methyl mercaptan, diphenyl sulfide, sulfoxides, sulfones, dimethyl sulfide, dimethyl trisulfide or combinations thereof.

In an embodiment, the VOC comprises ketones. For example and without limitation the VOCs may comprise acetone, butanone, 2-butanone, pentanone, hexanone, heptanone, or combinations thereof.

In an embodiment, the VOCs comprise metabolic indicators of oxidative stress such as nitrates, thromboxane, neopterin, n-acetylaspartate, myo-inositol and creatine.

It is contemplated that urinary metabolomic changes as a result of a transplantation rejection event and consequently VOCs other than those specifically identified in this disclosure may also be utilized to predict and/or diagnosis such an event. Thus an aspect of the present disclosure comprises the identification of urinary metabolomic changes that positively correlate to the prediction and/or detection of such an event.

It is contemplated that at least one VOC of the type disclosed herein may be utilized to predict and/or diagnose a transplant rejection event. In some embodiments, methods of predicting and/or diagnosing a transplant rejection event may utilize at least any number of VOCs where the number of VOCs utilized is represented as n and n is an integer ranging from 1 to 100, alternatively from 1 to 50, alternatively from 1 to 25, or alternatively from 1 to 10. In some embodiments, the relationship between the presence and/or amount of more than one VOC may be utilized to predict and/or diagnose a transplant rejection event. Such relationships may be determined by any suitable methodology.

In an embodiment, chemometric analysis is performed on the data obtained from a series of at least two biological samples of the type disclosed herein having different, known compositions, which are studied to ascertain interrelationships between the types and amounts of VOCs present and one or more known sample characteristics (e.g., sample was obtained from a subject experiencing a transplant rejection event). Such samples which have known characteristics are termed biological training samples. In an embodiment, chemometric analysis is carried out to ascertain the presence of interrelationships between VOCs present and a known sample characteristic using at least 5 biological training samples, or at least 10 biological training samples, or at least 20 biological training samples, or at least 30 biological training samples, or at least 40 biological training samples, or at least 50 biological training samples. The limit to the number of biological training samples that can be analyzed together to ascertain interrelationships between sample characteristics and outcomes (e.g., transplantation rejection event) usually is dictated by limitations of the software and computer hardware employed, and no specific upper limit to the number of samples to be used is contemplated.

Normally (as here), a range of biological training samples having different known compositions is tested so the differences in the data obtained for the respective samples can be evaluated to find changes in a pertinent dependent variable (e.g., VOC type and amount) arising from changes in an independent variable (e.g., physiological state of subject). One can, however, employ a set of biological training samples that include some duplicate, triplicate, or more redundant samples. The inclusion of redundant biological training samples in a set that also includes many diverse biological training samples may reduce the statistical error. Biological training samples optionally can be samples characterized in prior work, the literature, by interpolation or extrapolation from other training samples, or other sources, as opposed to biological training samples that are made physically available.

Another issue relating to the implementation of chemometric analysis to establish interrelationships between the VOC type and/or amount and the physiological state of the subject is the nature of the biological training samples selected. In an embodiment, biological training samples are selected to provide for the representation of a broad range of physiological states (e.g., early transplant rejection event, absence of transplant rejection event, transplant rejection event, post-transplant rejection event etc. . . . ) and the associated VOC type and/or amount. Analytical data for the biological training samples can be measured, obtained from literature values, derived from prior work, or obtained from a combination of sources. The information obtained on the VOC type and content for the biological training samples may be analyzed to find correlations between the predicted and/or diagnosed physiological state of the subject. Analysis of the relationship between the VOC type and amount and the predicted physiological state may be carried out using any suitable chemometric software. In an embodiment, the chemometric software compares the VOC type and amount in the biological samples and the physiological state of the subject.

In an embodiment, chemometric analysis of the relevant data is carried out using any suitable chemometric technique. Examples of suitable chemometric techniques include but are not limited to Partial Least Squares Regression (PLS), Multilinear Regression Analysis (MLR), Principal Components Regression (PCR), Principal Component Analysis (PCA), Multivariate Data Analysis, and Discriminant Analysis, as well as Design of Experiment (DOE) and Response Surface Methodologies. In an embodiment, the chemometric analysis is carried out using PLS2. PLS refers to a wide class of methods for modeling relations between sets of observed variables by means of latent variables. The underlying assumption of all PLS methods is that the observed data is generated by a system or process which is driven by a small number of latent variables.

In an embodiment, one or more of the VOCs disclosed herein may be detected in the biological sample in amounts ranging from 100 parts-per-trillion (ppt) to 100 parts-per-million (ppm), alternatively from 1 parts-per-billion (ppb) to 100 ppm, alternatively from 10 ppb to 100 ppm, alternatively in amounts greater than 10 ppb, alternatively in amounts greater than 50 ppb, alternatively in amounts greater than 100 ppb, alternatively in amounts greater than 1 ppm, alternatively in amounts greater than 10 ppm, or alternatively in amounts greater than 50 ppm. It is contemplated that these amounts may reflect the results of processing the biological samples to facilitate detection of the VOCs (e.g., concentration of breath and/or urine samples).

In an embodiment, the methodologies disclosed herein are able to predict a subject who in the absence of other symptoms has an increased propensity to experience a transplant rejection event. In an embodiment, the methodologies disclosed herein are able to diagnose a subject experiencing a transplant rejection event. For example, the International Society for Heart and Lung (IHST) grades the degree of transplant rejection utilizing a scale ranging from 0 to 4 where at Grade 0 there are no indications of rejection; at Grade 1 the rejection is mild; at Grade 2 the rejection is focal moderate; at Grade 3 the rejection is multifocal to borderline severe; and at Grade 4 the rejection is severe. In an embodiment, the methodologies disclosed herein may result in the identification of a transplant rejection event at Grades 1, 2, 3, or 4; alternatively at Grades 1, 2, or 3; alternatively at Grades 1 or 2; or alternatively at Grade 1. In an embodiment, the methodologies and devices disclosed herein may be advantageously utilized to predict and/or diagnose a transplant rejection event within 30 days of the organ transplant, or alternatively within 14 days, or alternatively within 7 days. In an embodiment, prediction and/or diagnose of a transplant rejection event utilizing the methodologies disclosed herein may be performed at an early degree of rejection (e.g., Grade 0 or Grade 1) allowing the subject to undergo one or more additional methodologies to mitigate the adverse events associated with the transplant rejection event. For example, the subject may be provided one or more therapies designed to inhibit or prevent the onset of adverse events associated with the transplant rejection event or may be able to reverse the transplant rejection event altogether.

In an embodiment disclosed herein, a subject having undergone an organ transplant may be subjected to health surveillance utilizing the methodologies disclosed herein. For example, a biological sample of the type disclosed herein (e.g., urine or breath) may be regularly collected from and/or by the subject and analyzed for the type and amount of VOCs present in order to provide a regular assessment of physiological state of the subject. For example, health surveillance may be carried out on a subject having undergone a cardiac allograft and the methodologies disclosed herein may provide information on the degree of rejection for the subject on a regular basis. For example the biological sample may be collected from every 24 hours to every 30 days, alternatively from every 24 hours to every 14 days, alternatively from every 24 hours to every 7 days, or alternatively from every 24 hours to every 48 hours and information on the degree of rejection provided in an identical timeframe. In some embodiments, information on the degree of rejection is available to the subject and/or healthcare provide on a regular basis. Such information may be provided in any suitable format (e.g., paper printout, digital media, electronically, etc. . . . ) to the subject and/or healthcare provider.

In an embodiment, the present disclosure further relates to a device configured for detecting the presence of the VOCs in aqueous or gaseous phases, referred to herein as an electronic nose (eN) device. In an embodiment, the eN device comprises one or more sensors configured to detect one or more VOCs, alternatively, to detect one or more groupings of VOCs, such as one or more of the VOC species disclosed herein.

In an embodiment of the present eN device, one or more of the sensors comprises a catalytic or semiconducting material comprising a metallic material. Examples of suitable metallic materials include but are not limited to metals, metal alloys, metal oxides, nanomaterials, and combinations thereof, without limitation. The sensor may be constructed by any metallic deposition method including but not limited to sputtering, chemical-vapor-deposition, or combinations thereof. In some configurations, the sensor comprises a plurality of layers deposited on a substrate. Each of the layers of the sensor may comprise different metals, metal alloys, metal oxides, nanomaterials, and combinations thereof. In certain configurations, the sensor is doped to preferentially detect one or more of the VOCs. Without limitation by any theory, doping a sensor may modulate the catalytic and/or semiconducting properties of the metallic material. In further configurations, each layer of a sensor may comprise a different dopant.

In an additional or alternative embodiment of the present eN device, one or more of the sensors comprises a microchip sensor. In such an embodiment, the microchip sensor may comprise a generally planar substrate generally configured such that the liquid or gaseous sample flows across the microchip sensor.

In an embodiment, the one or more sensors may be configured to output a signal indicative of the presence, absence, amount, or concentration of one or more VOCs, alternatively, of one or more groupings of VOCs. For example, in an embodiment, the one or more sensors may be configured to output a suitable electrical signal. In an embodiment, a suitable electrical signal may comprise a varying analog voltage or current signal, for example, representative of the presence, absence, amount, or concentration of one or more VOCs, alternatively, of one or more groupings of VOCs. In an alternative embodiment, the suitable electrical signal may comprise a digital encoded voltage signal. In an embodiment, the eN device may output the signal generated by one or more of the sensors (alternatively, a processed signal resulting therefrom) via a suitable communications link (e.g., a USB connection or a wireless link).

Additionally, in an embodiment, the eN device may be configured to process the signal output by one or more of the signals. For example, in an embodiment, the eN device may comprise an electronic circuit comprising a plurality of functional units. In an embodiment, a functional unit (e.g., an integrated circuit (IC)) may perform a single function, for example, serving as an amplifier or a buffer. The functional unit may perform multiple functions on a single chip. The functional unit may comprise a group of components (e.g., transistors, resistors, capacitors, diodes, and/or inductors) on an IC which may perform a defined function. The functional unit may comprise a specific set of inputs, a specific set of outputs, and an interface (e.g., an electrical interface, a logical interface, and/or other interfaces) with other functional units of the IC and/or with external components. In some embodiments, the functional unit may comprise repeat instances of a single function (e.g., multiple flip-flops or adders on a single chip) or may comprise two or more different types of functional units which may together provide the functional unit with its overall functionality. For example, a microprocessor or a microcontroller may comprise functional units such as an arithmetic logic unit (ALU), one or more floating-point units (FPU), one or more load or store units, one or more branch prediction units, one or more memory controllers, and other such modules. In some embodiments, the functional unit may be further subdivided into component functional units. A microprocessor or a microcontroller as a whole may be viewed as a functional unit of an IC, for example, if the microprocessor shares a circuit with at least one other functional unit (e.g., a cache memory unit). In some embodiments, the functional units may comprise resistive heating unit to refresh the sensor surface between the measurement of different samples. In some embodiments, the functional units may be used together with accessories of infrared heater and vacuum unit to refresh the sensor surface between the measurements of different samples.

The functional units may comprise, for example, a general purpose processor, a mathematical processor, a state machine, a digital signal processor (DSP), a receiver, a transmitter, a transceiver, a logic unit, a logic element, a multiplexer, a demultiplexer, a switching unit, a switching element an input/output (I/O) element, a peripheral controller, a bus, a bus controller, a register, a combinatorial logic element, a storage unit, a programmable logic device, a memory unit, a neural network, a sensing circuit, a control circuit, an analog to digital converter (ADC), a digital to analog converter (DAC), an oscillator, a memory, a filter, an amplifier, a mixer, a modulator, a demodulator, and/or any other suitable devices as would be appreciated by one of ordinary skill in the art.

In an embodiment, the eN device may be configured to retain a volume of fluid in proximity to the sensor. For example, in an embodiment, the eN comprises a channel or conduit for directing an aqueous or gaseous sample across or proximate to at least one sensor.

In an embodiment, the eN device comprises a plurality of sensors forming a sensor array. Without limitation, the sensor array may be configured such that any two of the one or more sensors are positioned in parallel, sequentially, coaxially, or combinations thereof. In an embodiment, the sensor array comprises a first sensor configured to detect the presence, absence, amount, or concentration of a first VOC (alternatively, a first grouping of VOCs) and a second sensor configured detect the presence, absence, amount, or concentration of a second VOC (alternatively, a second grouping of VOCs). For example, the sensor array may comprise various sensors responsive to various, differing VOCs or other bioindicators.

For example, in an embodiment, the eN comprises a tube-in-tube configuration, such that the sample travels from the inner tube to the outer tube across a sensor array, or vice versa. Alternatively, in an embodiment, the eN comprises a plurality of porous walls, such as a honeycomb structure, such that the sample progresses across a plurality of sensors as the sample passes through each porous wall. In certain configurations, the eN sensor array comprises a porous, or nano-porous material, such as a membrane. Further, the eN sensor may comprise a rough or textured surface to increase the contact area between the sample and the sensors. In certain configurations, the eN device may be configured to be dipped or temporarily submerged or coated in a sample, particularly a liquid sample.

In an exemplary embodiment, the eN device comprises a coaxial configuration. Without limitation by any theory, a sensor or sensor array is deposited on the inside of a cylindrical substrate. Alternatively, the sensor or sensor array is deposited on a flat or planar substrate that is subsequently rolled to form at least one cylindrical structure. The sensors are disposed at a fixed distance from the axis of the cylindrical shape, thus all sensors or sensor arrays in the eN device are co-axial. In this configuration, the gaseous or liquid sample is passed through the cube and contacts the sensor or sensor array.

Exemplary embodiments of the invention are disclosed herein and variations, combinations, and/or modifications of such embodiment(s) may be made by a person having ordinary skill in the art and are within the scope of the disclosure. Alternative embodiments that result from combining, integrating, and/or omitting features of the expressly-disclosed embodiment(s) are also within the scope of the disclosure. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, R₁, and an upper limit, Rᵤ, is disclosed, any number falling within the range is specifically disclosed. In particular, the following numbers within the range are specifically disclosed: R=R₁+k* (Rᵤ−R₁), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent . . . 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim means that the element is required, or alternatively, the element is not required, both alternatives being within the scope of the claim. Use of broader terms such as “comprises”, “includes”, and “having” means “including but not limited to” and should be understood to also provide support for narrower terms such as “consisting of”, “consisting essentially of”, and “comprised substantially of.” Accordingly, the scope of protection is not limited by the description set out above but is defined by the claims that follow, that scope including all equivalents of the subject matter of the claims. Each and every claim is incorporated as further disclosure into the specification, and each is an exemplary embodiment of the present invention. The discussion of a reference in the disclosure is not an admission that it is prior art, especially any reference that has a publication date after the priority date of this application. The disclosure of all patents, patent applications, and publications cited in the disclosure are hereby incorporated by reference, to the extent that they provide exemplary, procedural or other details supplementary to the disclosure.

To further illustrate various exemplary embodiments of the present invention, the following examples are provided.

### Examples

The present disclosure relates to non-invasive early diagnosis of the heart rejection.

A methodology of the type disclosed herein comprises at least two components: (1) the utilization of urine samples to recover VOCs that are produced through rejection-related abnormal metabolic pathways; and (2) a nanosensor based, highly sensitive, redundant and steadily structured electronic nose system.

VOC biomarkers of rejection animal model to optimize the sample preparation to efficiently extract VOC chemicals: Identify urinal VOCs produced by post-transplanted animals with GC-MS; To statistically screen the candidate markers and conduct internal and external validations; To conduct quality control and identify the VOC markers; To sort out longitudinal shift of the VOC signatures vs. the progress of rejection, in order to identify the markers at early stage rejection.

VOC signature pattern with the eN system to demonstrate proof-of concept of the early rejection diagnosis in animal model: Improve the sensor selectivity by engineering the material doping and imprint design; Set up an electronic nose system with sensor chips and standardize urine sample pretreatment and “sniffing” style with artificially formulated urine samples; recognize specific response patterns to rejection samples; and demonstrate a sensitive detection with the eN system for the early stage rejection.

Generally, blood flows into major organs like heart, lung, liver and kidney, then carry out these metabolites with abundant health information. Both breath and urine are primary windows for sampling and detecting. Breath analysis of C4-C20 alkanes and monomethylalkanes has derived breath methylated alkane contour (BMAC) as one marker for rejection. The metabolic pathway is related to oxidative stress that degrades membrane polyunsaturated fatty acids, and evolves alkanes and methylalkanes. In addition to rejection detection, VOC biomarkers identified in breath analysis with gas chromatography-mass spectrometry (GC-MS) have also presented their potentials to diagnose lung cancer, heart disease, pulmonary tuberculosis, liver cirrhosis, etc. Though, research has not been established concentrations and ratios for early diagnosis yet, the VOC strategy for health surveillance appears superior to others regarding the non-invasive and unlimited sampling. It poses no risk to patients to conduct longitudinal monitoring against early symptom of rejection on a daily basis.

The apparatus and methods of the present disclosure relate to a convenient and affordable detection system that can be easily operated and is intended to replace the expensive and complicated GC-MS systems. A rejection diagnosis system building upon the principles and disclosure herein presents a means to provide rejection treatment at its early stage, with an aim being improvement in patient survival rates and quality of life.

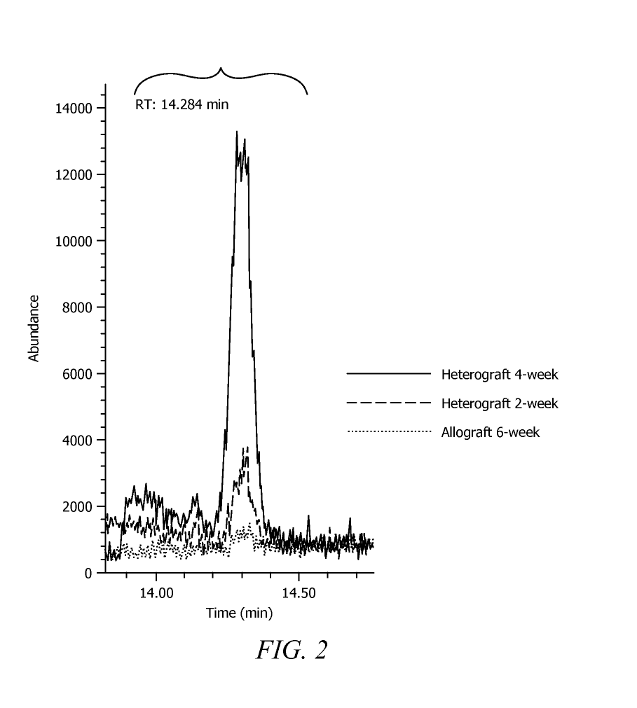

Preliminary GC-MS data with mouse urine samples showed distinctive VOC peaks as found in FIG. 2. Urinal samples from heart-transplanted mice were examined with GC-MS. At retention time 14.284 min, all transplanted mice have a diphenyl sulfide peak that of 93% match to the library of the National Institute of Standards and Technology (NIST). Moreover, the “Heterograft 4-week” that normally has rejection symptoms shows 3-fold higher abundance than the “Allograft 6-week”. It is also higher than “Heterograft 2-week” that represents the early stage of rejection. This result implicates the enhanced metabolism of the sulphur-containing compounds during the course of rejection. The result also appears to connect with the P450 involving immunity. Different from transcription, protein abundance and tissue changes, the metabolic changes typically happen within seconds or minutes after an ‘event’. The urinal VOC permits detection of such early events by its higher signal feature amplitude than that in the breath samples. The detection of the rejection event may be expanded from usually the inflammatory and oxidative stresses pathways to the T/B cell activation that preludes the pathogenic changes, in order to decipher the timely VOC signature transition for early diagnosis of the rejection.

A novel and sensitive electronic nose (eN) with the combination a novel nano-coaxial and molecular imprinted nanosensors is disclosed. The LOD of the disclosed sensor is sub-ppb for VOC, and pg/L for biomarker molecules. This sensitivity allows the detection of low concentration VOC biomarkers at the early stage of heart rejection, for example. The site density of the sensors is 10⁸/cm². Thereby, the redundancy in the new eN system exceeds certain previously developed systems. Also, the combination of molecular imprint, nanocoaxial structures, doping of the sensing materials and impedance spectroscopy provide tunable means to increase the specificity to the biomarker species to capture more sophisticate signature patterns to identify disease characters. The coaxial sensor disclosed herein can detect VOC concentrations spanning almost 5 orders of magnitudes or 5 decades. This accommodates the potentially large VOC fluctuation in the urine samples, so that the system can not only qualitatively discriminate the occurrence of rejection, but also quantitatively provide grading of the rejection event and the VOC concentrations.

METHOD: Benchmarks for this disclosure are: identification of urinal VOC biomarkers with more than 80% sensitivity and selectivity; and detect allo-rejection with the biomarkers in less than 1 week post transplantation and identify the biggest pitfalls related to the unknown composition of the urinal samples

Cardiac transplantation is performed with procedures as described previously. Allograft (HT −ve) and heterograft (HT +ve) are to produce samples. According the preliminary setting, each sample will be 1:10 diluted with 0.9% NaCl at pH7 to avoid foaming during VOC sampling with the purge-and-trap (Velocity XPT, Teledyne Tekmar). Initially, the samples are evaluated with the EPA Method 8260B for determining VOC with boiling temperature below 200° C. in complex aqueous matrixes. Additional treatments may be conducted to improve the VOC concentration and reduce the background interferences. For example, urease enzyme may be added to deplete excess urea, a chromatographic interference. A concentration of 30-100 U of urease enzyme may be used, dependent on the volume of urine sample. Acidification may also increase the number of identifiable metabolites in the urine samples by, for example by ˜37%. Less volatile metabolites such as organic acids, can be treated with N,O-bis-trimethylsilyl-trifluoroacetamide (BSTFA) or N-methyl-N-(trimethylsilyl)trifluoroacetamide) (MSTFA) for trimethylsily-1 derivatization. The reproducibility and accuracy of the analysis is configured to be inspected frequently with a mixture of standard compounds, the healthy control sample, or the healthy control sample in a cocktail of three standard solvents (tetradecane, pentadecane, and hexadecane at 0.218 g/L). Particularly, a “quality control” (QC) sample can be prepared by mixing equal volumes from the samples to be analyzed and aliquoted for analysis. It will provide a representative “mean” sample containing all the analytes that will be encountered during the analysis. During confirmation, the sample will be randomized to avoid biasness and overfitting of the data before the data acquisition with GC-MS. Different chemo-metric data mining strategies will be employed in the investigations. The data will be preprocessed with either ChemsStation (Agilent) or AMDIS (National Institute of Standards and Technology) for baseline correction, noise reduction, smoothing, missing value replacement, area calculation, and normalization. The missing values in the data table will be replaced with half a minimum value found in the data set, and total area normalization will be performed by dividing the integrated area of each analyte by the sum of total peak areas of analytes present in the sample. The converted data will be exported to SIMCA (Umtrics) for multivariate statistical analysis. Principal component analysis (PCA) will be used to visualize the grouping trends and outliers. Then, a supervised analysis technique, such as partial least squares discriminant analysis (PLS-DA) or orthogonal PLS-DA (OPLS-DA), will be used to build a model for the identification of the putative markers based on the discriminatory power. The model will be evaluated by internal and external validation. The internal validation of PLS-DA models will be performed iteratively (>900) using permutations test and receiver operating characteristic (ROC) analysis, according to the goodness of fit (R2 and Q2) and cross validation with predictions. The sensitivity and specificity are correlated to the area under the ROC curve. The validated model with sensitivity and specificity higher than 80% can be summarized and put for external validation with the pre-defined training set. The rejection predictability will be evaluated with the blinded set of samples. Putative marker VOC chemicals responsible for class separation are identified using variable importance plots (VIPs). The analytical variation related to each marker VOC is assessed by calculating its relative standard deviation (% r.s.d.) of the QC sample. It will be used to assess whether or not the identification of the marker metabolite is due to chance correlation. If a marker VOC is found with variation >30%, chemometric analysis will be reevaluated after removing this molecule. Also, if any of the marker VOCs are interfered with column bleed, reagent artifacts and xenobiotics, these molecules need to be removed from the data sets and chemometric models will be re-evaluated. In addition, for all the candidate markers, peak integration and library matching will be verified by interrogating the raw data manually. Their statistical significance will be evaluated using univariate statistical tests such as unpaired t-test and Welch's t-test. Considering the standard of isomers may not be commercially available, the identification of metabolites will be done not only relying on EI spectral similarity, but also the retention indices (RIs), in order to optimize the quality and reliability of library hits. The Golm metabolite library and the Human Metabolome Database (HMDB) can be cross-referenced for the characterization. Comparing to biomarkers derived by genomics, proteomics and histology that normally present over days or weeks, metabolic changes typically happen within seconds or minutes after an “event”.

The following are enumerated embodiments which are provided as non-limiting examples:

A first embodiment which is a method comprising (a) obtaining one or more biological samples from a subject wherein the subject has undergone an organ transplant; (b) determining the amount and type of one or more volatile organic compounds in the biological sample; and (c) correlating the amount of volatile organic compounds to a degree of transplant rejection.

A second embodiment which is the method of the first embodiment wherein the biological sample is selected from the group consisting of urine, blood, and breath.

A third embodiment which is the method of any of the first through second embodiments wherein the volatile organic compounds comprise hydrocarbons, alcohols, sulfides, sulfoxides, sulfones, aldehydes, ketones, nitriles, aromatics, organic acids, nitrates, thromboxane, neopterin, n-acetylaspartate, myo-inositol, creatine or combinations thereof.

A fourth embodiment which is the method of the third embodiment wherein the hydrocarbons comprise C₁ to C₂₀ aliphatic hydrocarbons.

A fifth embodiment which is the method of the third embodiment wherein the hydrocarbons comprise methylated alkanes.

A sixth embodiment which is the method of the third embodiment wherein the hydrocarbons comprise 2-methylpropane, 5-methyloctadecane, 6-methyloctadecane, 2-methylheptadecane, 2-methylheptane, 3-methylundecane, 2-methyloctadecane, 2-methylhexadecane, or combinations thereof.

A seventh embodiment which is the method of the third embodiment wherein the organic acids comprise hippuric acid, methyl succinic acid, mandelic acid, muconic acid, fuoric acid, butyric acid, hydroxybutryic acid, derivatives thereof or combinations thereof.

An eighth embodiment which is the method of the third embodiment wherein the sulfides comprise hydrogen sulfide, methyl mercaptan, diphenyl sulfide, dimethyl sulfide, dimethyl trisulfide or combinations thereof.

A ninth embodiment which is the method of the third embodiment wherein the ketones comprise acetone, butanone, 2-butanone, pentanone, hexanone, heptanone, or combinations thereof.

A tenth embodiment which is the method of any of the first through ninth embodiments wherein the correlating the amount of volatile organic compound to degree of transplantation comprises chemometric analysis.

An eleventh embodiment which is the method of any of the first through tenth embodiments wherein the volatile organic compound is present in the biological sample in an amount ranging from about 100 ppt to about 100 ppm.

A twelfth embodiment which is the method of any of the first through eleventh embodiments further comprising predicting the degree of transplant rejection.

A thirteenth embodiment which is a device comprising a plurality of sensors configured to detect a plurality of volatile organic compounds in a biological sample of a subject having undergone an organ transplant.

A fourteenth embodiment which is the device of the thirteenth embodiment wherein the volatile organic compounds are in the gaseous state, the liquid state, or a combination thereof.

A fifteenth embodiment which is the device of any of the thirteenth through fourteenth embodiments wherein one or more of the sensors comprises a catalytic or semiconducting material.

A sixteenth embodiment which is the device of the fifteenth embodiment wherein the catalytic or semiconducting material comprises s metal, a metal an alloy, a metal oxide, a nanomaterial, or combinations thereof.

A seventeenth embodiment which is the device of any of the thirteenth through sixteenth embodiments wherein one or more of the sensors comprises a microchip.

An eighteenth embodiment which is the device of any of the thirteenth through seventeenth embodiments configured to output one or more signals indicating the presence of one or more volatile organic compounds.

A nineteenth embodiment which is the device of any of the thirteenth through eighteenth embodiments configured to output one or more signals indicating the amount of one or more volatile organic compounds.

A twentieth embodiment which is the device of any of the thirteenth through nineteenth embodiments configured to output an electrical signal.

A twenty-first embodiment which is the device of any of the thirteenth through twentieth embodiments further comprising an electronic circuit wherein the electronic circuit comprises a plurality of functional units.

A twenty-second embodiment which is the device of any of the thirteenth through twenty-first embodiments wherein the plurality of sensors form a sensor array.

What is claimed is:

1. A method comprising
(a) obtaining one or more biological samples from a subject wherein the subject has undergone an organ transplant;
(b) determining the amount and type of one or more volatile organic compounds in the biological sample; and
(c) correlating the amount of volatile organic compounds to a degree of transplant rejection.

2. The method of claim 1 wherein the biological sample is selected from the group consisting of urine, blood, and breath.

3. The method of claim 1 wherein the volatile organic compounds comprise hydrocarbons, alcohols, sulfides, sulfoxides, sulfones, aldehydes, ketones, nitriles, aromatics, organic acids, nitrates, thromboxane, neopterin, n-acetylaspartate, myo-inositol, creatine or combinations thereof.

4. The method of claim 3 wherein the hydrocarbons comprise C₁ to C₂₀ aliphatic hydrocarbons.

5. The method of claim 3 wherein the hydrocarbons comprise methylated alkanes.

6. The method of claim 3 wherein the hydrocarbons comprise 2-methylpropane, 5-methyloctadecane, 6-methyloctadecane, 2-methylheptadecane, 2-methylheptane, 3-methylundecane, 2-methyloctadecane, 2-methylhexadecane, or combinations thereof.

7. The method of claim 3 wherein the organic acids comprise hippuric acid, methyl succinic acid, mandelic acid, muconic acid, fuoric acid, butyric acid, hydroxybutryic acid, derivatives thereof or combinations thereof.

8. The method of claim 3 wherein the sulfides comprise hydrogen sulfide, methyl mercaptan, diphenyl sulfide, dimethyl sulfide, dimethyl trisulfide or combinations thereof.

9. The method of claim 3 wherein the ketones comprise acetone, butanone, 2-butanone, pentanone, hexanone, heptanone, or combinations thereof.

10. The method of claim 1 wherein the correlating the amount of volatile organic compound to degree of transplant rejection comprises chemometric analysis.

11. The method of claim 1 wherein the volatile organic compound is present in the biological sample in an amount ranging from about 100 ppt to about 100 ppm.

12. The method of claim 1 further comprising predicting the degree of transplant rejection.

13. A device comprising a plurality of sensors configured to detect a plurality of volatile organic compounds in a biological sample of a subject having undergone an organ transplant.

14. The device of claim 13 wherein the volatile organic compounds are in the gaseous state, the liquid state, or a combination thereof.

15. The device of claim 13 wherein one or more of the sensors comprises a catalytic or semiconducting material.

16. The device of claim 15 wherein the catalytic or semiconducting material comprises s metal, a metal alloy, a metal oxide, a nanomaterial, or combinations thereof.

17. The device of claim 13 wherein the sensor comprises a microchip.

18. The device of claim 13 configured to output one or more signals indicating the presence of one or more volatile organic compounds.

19. The device of claim 13 configured to output one or more signals indicating the amount of one or more volatile organic compounds.

20. The device of claim 13 configured to output an electrical signal.

21. The device of claim 13 further comprising an electronic circuit wherein the electronic circuit comprises a plurality of functional units.

22. The device of claim 13 wherein the plurality of sensors form a sensor array.
